(19) United States
(12) Patent Application Publication
(10) Pub. No.: US 2007/0020197 A1
(43) Pub. Date: Jan. 25, 2007

(54) Preparation of pharmaceutical compositions containing nanoparticles

(75) Inventors: Christopher J. Galli, Ann Arbor, MI (US); Mayur P. Lodaya, (US); Matthew J. Mollan JR., Northville, MI (US); William Michael Polak, (US); Umang Shah, Ann Arbor, MI (US); Chandra Vemavarapu, Westland, MI (US)

Correspondence Address: WARNER-LAMBERT COMPANY, 2800 PLYMOUTH RD, ANN ARBOR, MI, 48105, US

(73) Assignee: WARNER-LAMBERT COMPANY LLC

(21) Appl. No.: 11/170,036

(22) Filed: Jun. 29, 2005

Related U.S. Application Data
(60) Provisional application No. 60/585,411, filed on Jul. 1, 2004.

(51) Int. Cl. A61L 9/04; A61M 11/00; A61K 9/14

(52) U.S. Cl. 424/46; 128/200.23

(57) ABSTRACT

Materials and methods for preparing pharmaceutical nanoparticle suspensions or dispersions, granulations and dosage forms are disclosed. The methods employ a modular high-pressure spray homogenizer coupled to a wet granulator to form stabilized nanoparticle suspensions and granulations.

### CROSS-REFERENCE TO RELATED APPLICATIONS

This application claims priority from U.S. Provisional Patent Application No. 60/585,411 filed Jul. 1, 2004.

### BACKGROUND OF THE INVENTION

1. Field of Invention

This invention relates to pharmaceutical compositions containing nanoparticles, and to methods and materials for preparing stable nanoparticulate suspensions, granulations, and dosage forms.

2. Discussion

The dissolution rate of a drug is a function of its intrinsic solubility and its particle size. Studies with poorly soluble drugs have demonstrated that particle size reduction can lead to an increased rate of dissolution and higher bioavailability. See R. H. Muller, Proceed. Int'l Symposium Control Rel Bioact Matter, Controlled Release Society, Inc 25 (1998) and U.S. Pat. No. 5,399,363 to G. G. Liversidge et al. The majority of these studies involve mechanical size reduction of particles to sizes larger than 1 μm. See, e.g., D. E. Englund & E. D. Johansson, Ups. J. Med. Sci. 86:297-307 (1981); J. T. Hargrove et al., Am. J. Obstet. Gynecol. 161:948-51 (1989); and S. Shastriet al., Am. J. Vet. Res. 41:2095-101 (1980). Researchers have reported a doubling in bioavailability of an anti-tumor agent, HO-221, when its mean particle size is reduced from 4.15 μm to 0.45 μm. See Kondo et al., Bio Pharm Bull 16:796-800 (1993). These studies suggest that there is considerable potential for substantially enhancing bioavailability by particle size reduction to the submicron range. Indeed, a comparison of the absolute bioavailability of a nanoparticulate donazol formulation (82.3%) and an aqueous suspension of conventional donazol particles (5.1%) indicates that the use of a nanoparticle dispersion may overcome the dissolution rate limited bioavailability observed with conventional suspension of donazol. See G. G. Liversidge & K. C. Cundy, International Journal of Pharmaceutics 125(1):91-97 (1995).

Nanoparticulate technology offers a potential path to rapid preclinical assessment of poorly soluble drugs. It offers increased bioavailability, improved absorption, reduced toxicity, and the potential for drug targeting. See C. Jacobs et al., Int. J. Pharm. 196:161-64 (2000). Nanoparticulate technology may thus allow for the successful development of poorly water-soluble discovery compounds, as well as the revitalization of marketed products through improvements in dosing. Because of the high adhesiveness of nanoparticles on biological surfaces (e.g., epithelial gut wall), nanoparticulate technology may prolong the absorption time of poorly soluble drugs, thereby improving bioavailability. Additionally, the use of nanoparticulates may reduce gastric irritation associated with NSAIDs (non-steroidal anti-inflammatory drugs) and, perhaps, hasten their onset of action. See, e.g., U.S. Pat. No. 5,518,738 to W. M. Eickhoff et al. Nanosuspensions may eliminate or reduce the need for potentially irritating solubilizing agents and may provide higher loading for reduced injection volume in parenteral dosage forms. They also appear suitable for colonic delivery for treatment of colon cancer, helminth and other bacterial and parasitic infections, gastrointestinal inflammation, or other diseases associated with the gastrointestinal tract. See R. H. Muller et al., Advanced Drug Delivery Reviews 47:3-19 (2001) and V. Labhasetwar, Pharmaceutical News 4(6) (1997). Several nanoparticulate drug delivery systems for dosing antineoplastic agents, vaccines, insulin, and propranol (β-blocker) are in preclinical or clinical stages of development; two nanoparticle based drug delivery systems are registered for use in United States.

Several techniques have been employed for preparing nanoparticles, including wet milling and piston gap homogenization, each with varying degrees of success. For discussions related to wet milling, see, e.g., U.S. Pat. No. 5,518,187 to J. A. Bruno et al.; U.S. Pat. No. 5,862,999 to D. A. Czekai and L. P. Seaman; and U.S. Pat. No. 5,534,270 to L. De Castro; for discussions related to piston gap homogenization, see R. H. Muller & K. Peters, Int. J. Pharm. 160:229-37 (1998); K. P. Krause & R. H. Muller, Int. J. Pharm. 214:21-4 (2001); U.S. Pat. No. 5,543,133 to J. R. Swanson et al.; U.S. Pat. No. 5,858,410 to R. H. Muller et al.; U.S. Patent Application Ser. No. 2003/0072807 A1 to J. C-T. Wong et al.; and U.S. Pat. No. 5,510,118 to H. W. Bosch et al., the complete disclosures of which are herein incorporated by reference.

Wet milling is a simple, well understood process, which relies on impact and shear forces to reduce particle size. However, wet milling suffers from numerous disadvantages that limit its usefulness, including erosion, discoloration, fractionation, filteration, long processing times, low solids concentration, heat generation, and risk of bacterial growth requiring depyrogenation.

Piston gap homogenization, which utilizes cavitation forces and impact or shear forces to reduce particle size, appears to overcome some of the problems associated with wet milling. However, piston gap homogenization is not without problems. For instance, piston gap homogenization often requires preprocessing to adequately reduce particle size. See U.S. Patent Application Ser. No. 2002/0168402 to J. E. Kipp et al. (microprecipitation) and C. Jacobs & R. H. Muller, Pharmaceutical Research 19(2):189-94 (Feb. 2002) (pre-milling using a jet mill or hammer mill). In addition, piston gap homogenization typically requires low suspension viscosity, and it generates high impact forces that may lead to excessive wear of the homogenizer and concomitant heavy metal contamination of the product.

In addition, piston gap homogenization is unable to process nanoparticle suspensions having a solids loading greater than about 10% (w/w) and can usually only operate up to about 30,000 psig, which limits process throughput and particle size distribution. See, e.g., R. Bodmeier & H. Chen, J. Cont. Rel. 12:223-33 (1990); C. Jacobs & R. H. Muller, Pharmaceutical Research 19(2):189-94 (Feb. 2002); A. Calvor & B. Muller, Pharmaceutical Development & Technology 3(3):297-305 (1998); H. Talsma et al., Drug Develop. Ind. Pharm. 15(2):197-207 (1989); R. H. Muller et al., Proc 1^{st} World Meeting APGI/APV, Budapest 9/11 (May 1995); R. H. Muller et al., Int. J. Pharm. 196:169-72 (2000); German Patent Application No. DE4440337 A1 to R. H. Muller et al.; and U.S. Patent Application Ser. No. 2003/0072807 A1 to J. C-T. Wong et al.

The present application is directed to overcoming or at least reducing the effects of one or more of the problems set forth above.

### SUMMARY OF THE INVENTION

The present invention provides methods and materials for preparing pharmaceutical compositions containing nanoparticles, including stable nanoparticulate suspensions (or dispersions), granulations, and dosage forms. The claimed methods and materials provide significant advantages over existing nanoparticle technologies. The present invention employs a high-pressure spray (et) homogenizer to form nanoparticle suspensions (nanosuspensions), which are subsequently stabilized via wet granulation. Unlike wet milling or piston gap homogenization, the high pressure spray homogenizer is capable of independently controlling impact, cavitation, and shear forces, as well as flow characteristics (turbulent or laminar) to accommodate different solid fracture characteristics. Additionally, the system avoids many of the disadvantages associated with wet milling and piston gap homogenization, and is thus able to prepare nanosuspensions with minimal preprocessing and having solids concentrations as high as about 80% (w/w). The high solids loading of the nanosuspensions obviates the need for drying the nanosuspension and permits direct granulation of the solid dispersion.

One aspect of the present invention provides a system for preparing a pharmaceutical granulation. The system comprises a high-pressure spray homogenizer that is adapted to receive an active pharmaceutical ingredient and a liquid carrier, and to discharge a dispersion. The high-pressure spray homogenizer is configured to comminute the active pharmaceutical ingredient into solid particles having a median particle size of about 1 μm or less based on volume and to disperse the solid particles in the liquid carrier so as to form the dispersion. The solid particles comprise more than 2% w/w of the dispersion. The system also includes a granulator, which is in fluid communication with the high-pressure spray homogenizer and with one or more sources of pharmaceutically acceptable excipients. The granulator is configured to receive the dispersion from the high-pressure spray homogenizer and to combine the dispersion with the one or more pharmaceutical excipients so as to form the pharmaceutical granulation. Suitable granulators include twin-screw mixers and spray dryers.

Another aspect of the present invention provides a method of preparing a pharmaceutical granulation. The method comprises comminuting an active pharmaceutical ingredient into solid particles in the presence of a liquid carrier so as to form a dispersion. The solid particles have a median particle size of about 1 μm or less based on volume and they are substantially insoluble in the liquid carrier at room temperature. The method also includes combining the dispersion with one or more pharmaceutically acceptable excipients in a granulator so as to form a pharmaceutical granulation. The method optionally includes drying the pharmaceutical granulation.

Yet another aspect of the present invention provides a method of preparing a pharmaceutical dispersion. The method comprises comminuting an active pharmaceutical ingredient into particles in the presence of a liquid carrier. The active pharmaceutical ingredient is a solid at room temperature and it comprises more than 2% w/w of the pharmaceutical dispersion. Moreover, the particles that are dispersed in the liquid carrier have a median particle size of about 1 μm or less based on volume.

Still another aspect of the present invention provides a pharmaceutical dispersion. The pharmaceutical dispersion comprises an active pharmaceutical ingredient, which includes particles having a median particle size of about 1 μm or less based on volume. Other components of the pharmaceutical dispersion include a liquid carrier, and an optional surfactant. The active pharmaceutical ingredient is a solid, is substantially insoluble in the liquid carrier at room temperature, and comprises more than 2% w/w of the pharmaceutical dispersion.

A further aspect of the present invention provides a method of making a pharmaceutical dosage form. The method comprises comminuting an active pharmaceutical ingredient into solid particles in the presence of a liquid carrier so as to form a dispersion. The solid particles have a median particle size of about 1 μm or less based on volume. The method also includes combining the dispersion with one or more pharmaceutically acceptable excipients in a granulator so as to form a granulation. Optional steps include drying the granulation, milling the dried granulation, and combining the granulation (whether milled or not) with one or more pharmaceutically acceptable excipients.

An additional aspect of the present invention provides a method of making a pharmaceutical dosage form. The method includes comminuting an active pharmaceutical ingredient into solid particles in the presence of a liquid carrier so as to form a dispersion. The solid particles have a median particle size of about 1 μm or less based on volume, they are substantially insoluble in the liquid carrier at room temperature, and they comprise more than 2% w/w of the dispersion. The method also includes combining the dispersion with one or more pharmaceutically acceptable excipients.

In the inventive systems, methods, pharmaceutical dispersions and dosage forms, the solid particles typically comprise up to about 5% w/w or more, 10% w/w or more, 20% w/w or more, 30% w/w or more, 40% w/w or more, 50% w/w or more, 60% w/w or more, 70% w/w or more of the dispersion, or up to about 80% w/w of the pharmaceutical dispersion. Furthermore, useful granulators include twin-screw mixers and spray dryers.

### BRIEF DESCRIPTION OF THE DRAWINGS

FIG. 1 depicts a schematic of a system for preparing pharmaceutical nanoparticulate suspensions or dispersions, granulations, and dosage forms.

FIG. 2 depicts a modular high-pressure spray homogenizer for preparing nanoparticululate solids comprised of one or more active pharmaceutical ingredients dispersed or suspended in a continuous liquid phase.

FIG. 3 shows a Haake TSM screw design used in the Examples.

FIG. 4 shows photomicrographs that were obtained using an optical microscope and which illustrate the effect of the number of cycles on particle size of CPD-1 dispersions (TD0790503).

FIG. 5 shows particle size distribution of CPD-1 dispersions for different processing times (laser diffraction data, TD0790503).

FIG. 6 shows particle size distribution of naproxen dispersions for different processing times (laser diffraction data, TD0900703).

FIG. 7 shows d90, based on volume, for CPD-1 and naproxen dispersions as a function of processing time (TD0790503 and TD0900703).

FIG. 8 shows d10, d50, and d90, based on volume, of CPD-1 dispersions as a function of operating pressure for different backpressures (laser diffraction data, TD0450303).

FIG. 9 shows d10, d50, and d90, based on volume, of CPD-1 dispersions as a function of the number of cycles for different backpressures (laser diffraction data, TD0560403).

FIG. 10 and FIG. 11 show photomicrographs that were obtained using an optical microscope and which illustrate the effect of operating pressure and backpressure on particle size of CPD-1 (TD00450303).

FIG. 12 shows differential mass distribution of CPD-1 dispersions for two different backpressures (0 and 1 kpsig) (TD0560403).

FIG. 13 shows d10, d50, and d90, based on volume, of CPD-1 dispersions having solids concentrations of 1% and 10% (w/w) (TD0680503 and TD0710503).

FIG. 14 shows d10, d50, and d90, based on volume, of CPD-1 dispersions for different types of temperature control (TD0680503 and TD0710503).

FIG. 15 shows d90, based on volume, of CPD-1 dispersions as a function of surfactant concentration (laser diffraction data, TD0680503, TD0690503, and TD0700503).

FIG. 16 shows dissolution profiles of nanoparticulate and coarse dispersions of CPD-1 (TD0790503).

FIG. 17 shows dissolution profiles of nanoparticulate and coarse dispersions of naproxen (TD 0980803 and TD0990803).

FIG. 18 shows dissolution profiles of a tablet containing a nanoparticulate dispersion of naproxen and a commercially available formulation (Naprosyn®) at pH6.

FIG. 19 shows dissolution profiles of a tablet containing a nanoparticulate dispersion of naproxen and a commercially available formulation (Naprosyn®) at pH7.4.

FIG. 20 shows dissolution profiles of tablets containing a nanoparticulate dispersion of CPD-1 and those containing micronized CPD-1 or solid dispersions of CPD-1 in PVP or PVP and Tween 80.

FIG. 21 shows d10, d50, and d90, based on volume, of celecoxib dispersions as a function of the number of cycles (photon correlation spectrophotometer data, 8626×101).

FIG. 22 is a scanning electron photomicrograph of celecoxib nanoparticle dispersion.

### DETAILED DESCRIPTION

Definitions and Abbreviations

Unless otherwise indicated, this disclosure uses definitions provided below.

“About” or “approximately,” and the like, when used in connection with a numerical value, generally refers to a range of values that is ±10% of the stated value. Thus, for example, a median particle size of 100 μm would include median particle sizes within a range of 90 μm to 110 μm, inclusive.

“Particle size” refers to the median or the average dimension of particles in a sample and may be based on the number of particles, the volume of particles, or the mass of particles, and may be obtained using any number of standard measurement techniques, including laser diffraction methods, centrifugal sedimentation techniques or photon correlation spectroscopy (dynamic light scattering or quasi-elastic light scattering). Unless stated differently, all references to particle size in this specification refer to the median particle size based on volume, which may be obtained from measurements using a Coulter LS 230 Particle Size Analyzer (laser diffraction), CPS Instruments, Inc Disc Centrifuge Model DC18000 (centrifugal sedimentation), or Brookhaven 90 Plus Particle Size Analyzer (photon correlation spectroscopy).

“Dispersion” refers to finely divided particles distributed in a carrier or dispersion medium. In general, the particulate (dispersed) phase and the carrier medium (continuous phase) may be solids, liquids, or gaseous, but unless stated differently or otherwise clear from the context of the discussion, dispersion as used herein refers to solid particles dispersed in a solid, liquid, or gas carrier.

“Coarse dispersion” refers to a dispersion of particles in which the particles range in size from about 1 μm to about 500 μm.

“Nanoparticles,” “Nanoparticulates,” and the like, refer to discrete solid particles having a median particle size and d90, based on volume, less than about 1 μm and 5 μm, respectively, and more particularly, to particles having a median particle size and d90, based on volume, less than about 500 nm and 1 μm, respectively.

“Nanosuspensions,” “Nanodispersions,” and the like, refer to finely divided nanoparticles or nanoparticulates dispersed in a carrier or continuous medium. The carrier may be a liquid, solid, or gas, but is ordinarily a liquid or solid.

“Pharmaceutically acceptable” refers to substances, which are within the scope of sound medical judgment, suitable for use in contact with the tissues of patients without undue toxicity, irritation, allergic response, and the like, commensurate with a reasonable benefit/risk ratio, and effective for their intended use.

“Room temperature” refers to a temperature between about 20° C. and about 25° C., inclusive.

“Treating” refers to reversing, alleviating, inhibiting or slowing the progress of, or preventing a disorder or condition to which such term applies, or to preventing one or more symptoms of such disorder or condition. “Treatment” refers to the act of “treating.”

“Excipient” or “adjuvant” refers to any component of a pharmaceutical composition that is not the drug substance.

“Drug,” “drug substance,” “active pharmaceutical ingredient,” and the like, refer to a compound that may be used for treating a patient in need of treatment.

“Drug product,” “final dosage form,” and the like, refer to the combination of drug substance and excipients that are administered to a patient in need of treatment, and may be in the form of tablets, capsules, liquid suspensions, patches, and the like. The drug substance is present in a therapeutically effective amount for treatment of the patient.

“Poorly soluble” compounds include those that are classified as either “sparingly soluble,” “slightly soluble,” “very slightly soluble,” or “practically insoluble” in the United States Pharmacopoeia (USP), i.e., compounds having a solubility of one part of solute to about 30-100 parts of solvent, about 100-1000 parts of solvent, about 1000-10,000 parts of solvent, or about 10,000 or greater parts of solvent, respectively, when measured at room temperature and a pH between 2 and 12. Alternatively, poorly soluble compounds include those having a dose to aqueous solubility ratio greater than about 100 at a pH of about 5 to about 7.

TABLE 1 lists abbreviations used throughout the specification.

TABLE 1
List of Abbreviations

| Abbreviation | Description |
|---|---|
| ACN | acetonitrile |
| API | active pharmaceutical ingredient |
| COX | cyclooxygenase |
| CTAB | cetyltrimethylammonium bromide |
| d10, d50, d90 | cumulative distribution functions in which 10%, 50% and |
| | 90% of the solids (based on volume) have diameters less |
| | than d10, d50, and d90, respectively |
| DMSO | dimethylsulfoxide |
| EtOH | ethanol |
| HPC | hydroxypropyl cellulose |
| HPMC | hydroxypropyl methyl cellulose |
| HPS | high pressure spray |
| ID | inner diameter |
| IPA | isopropanol |
| MEK | methyl ethyl ketone |
| MeOH | methanol |
| PBO | polybutyl oxide |
| PEO | polyethylene oxide |
| pK | pharmacokinetic |
| psig | pounds per square inch (gauge) |
| PVP | polyvinylpyrrolidone |
| SLS | sodium lauryl sulfate |
| TSM | twin-screw mixer |
| USP | United States Pharmacopoeia |
| v/v | volume/total volume × 100, % |
| w/v | weight (mass) of solute/solvent volume × 100, % |
| w/w | weight (mass)/total weight (mass) × 100, % |

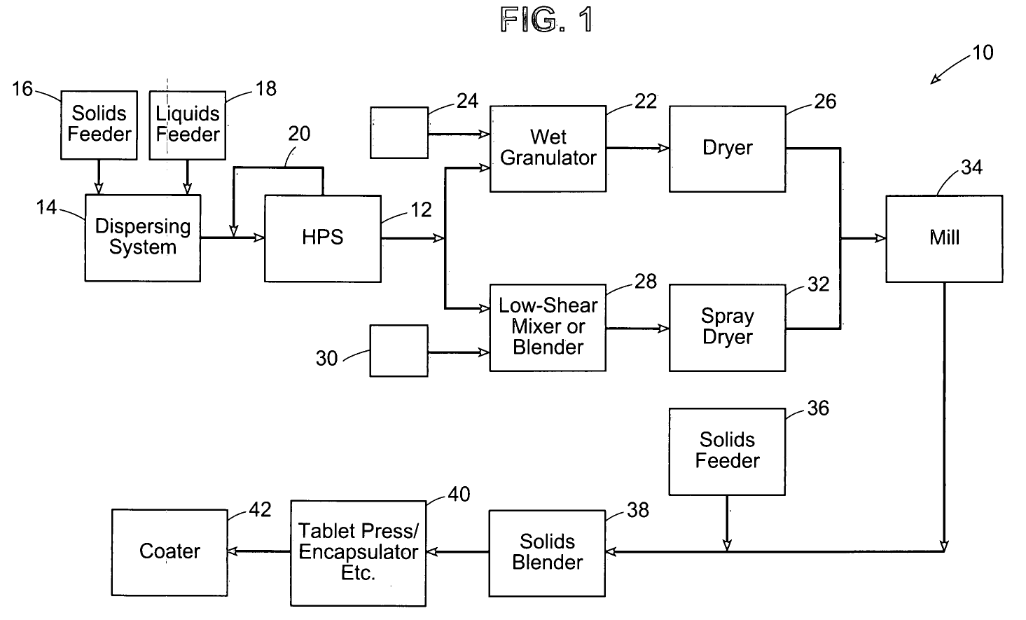

FIG. 1 depicts a schematic of a system 10 for continuously preparing pharmaceutical nanoparticulate dispersions or suspensions, granulations, and final dosage forms. The system 10 includes a modular high-pressure spray (jet) homogenizer 12, which is described in greater detail below. Unlike wet milling or piston gap homogenization, the high pressure spray (HPS) homogenizer 12 is capable of independently controlling impact, cavitation, and shear forces, as well as flow characteristics (turbulent or laminar) to accommodate different solid fracture characteristics of the active pharmaceutical ingredient (API).

As shown in FIG. 1, a solid-liquid dispersing system 14 (e.g., mixing vessel, colloid mill, etc.) supplies the high-pressure spray homogenizer 12 with one or more APIs. At least one of the active pharmaceutical ingredients is in the form of a coarse dispersion of discrete solid particles distributed or suspended in a continuous phase, which is usually a liquid, but may be a gas. For drugs having poor aqueous solubility, the liquid carrier is usually water; for other drugs, the liquid carrier is one or more organic “solvents” in which the drug is poorly soluble. These may include protic carriers (e.g., an alkanol such as EtOH, IPA, etc.), polar aprotic carriers (e.g., acetone, MEK, ACN, THF, DMSO, etc.), non-polar carriers (alkanes, such as hexanes, or aromatic, such as toluene), and the like. The coarse dispersion has a total solids loading of about 1% to about 80% (w/w). Material feeders 16, 18 provide the dispersing system 14 with the requisite solid and liquid components of the coarse dispersion, respectively. The system 10 generally includes a cooling system (not shown) for controlling the process temperature of the high-pressure spray homogenizer 12.

Besides the API and the carrier, the solid and liquid components of the coarse dispersion may include processing and dispersing aids (surfactants and stabilizers) and other excipients found in pharmaceutical dosage forms. These excipients may include, without limitation, low melting ethylene oxides (PEOs); oils, such as arachis oil, cottonseed oil, sunflower oil, and the like; semisolid lipophilic vehicles, such as hydrogenated specialty oils, cetyl alcohol, stearyl alcohol, gelucires, glyceryl behenate, and the like; solubilizing or emulsifying agents, such as Tween 80, SLS, CTAB, sodium deoxycholate, Imwitor, Cremophor, Poloxamer, and the like; and surface stabilizers, including cetyl pyridinium chloride, gelatin, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, sodium dodecylsulfate, carboxymethylcellulose calcium, hydroxypropyl celluloses, hydroxypropyl methylcellulose, hydroxypropylcellulose, carboxymethylcellulose sodium, methylcellulose, hydroxyethylcellulose, hydroxypropylmethyl-cellulose phthalate, noncrystalline cellulose, polyvinyl alcohol, polyvinylpyrrolidone, 4-(1,1,3,3-tetramethylbutyl)-phenol polymer with ethylene oxide and formaldehyde, poloxamers, poloxamines, dimyristoyl phophatidyl glycerol, dioctylsulfosuccinate, dialkylesters of sodium sulfosuccinic acid, sodium lauryl sulfate, an alkyl aryl polyether sulfonate, a mixture of sucrose stearate and sucrose distearate, p-isononylphenoxypoly -(glycidol), block copolymers of ethylene oxide and propylene oxide, and triblock copolymers of the structure —(—PEO)—(—PBO—)—(—PEO—)— and having a molecular weight (number average) of about 5000, and the like. Many of these surface stabilizers are known pharmaceutical excipients and are described in the Handbook of Pharmaceutical Excipients, published jointly by the American Pharmaceutical Association and The Pharmaceutical Society of Great Britain (1986), which is herein incorporated by reference. The surface stabilizers are commercially available or may be prepared by known techniques.

The coarse dispersion generally includes about 0.01 to about 10% w/w of one or more surfactants, and often includes about 0.1 to about 3% w/w of surfactants. In addition, the coarse dispersion generally includes about 0 to about 30% w/w of one or more surface stabilizers and often includes about 0 to about 12% w/w of surface stabilizers. In many cases, the coarse dispersion includes about 0 to about 8% w/w of surface stabilizers. The HPS homogenizer shown in FIG. 1 usually requires substantially less surfactant and stabilizer than systems that utilize attrition milling and piston gap homogenization.

As shown in FIG. 1, the course dispersion passes through the high-pressure spray homogenizer 12, where it forms a nanoparticulate dispersion or nanosuspension. A portion of the nanosuspension may optionally be reprocessed via a recycle loop 20, while the remainder of the nanosuspension is stored or, ideally, directly fed to a high-shear, wet granulator 22. One or more feeders 24 supply the wet granulator 22 with pharmaceutically acceptable excipients, which help stabilize the nanosuspension. The resulting wet granulation of stabilized nanoparticles enters a dryer 26 (e.g., a convective heat dryer, such as a fluid bed dryer, a radiant heat dryer, such as an IR tunnel dryer, and the like), which removes any residual liquid.

Alternatively, the nanosuspension exiting the HPS homogenizer 12 may be combined in a low-shear mixer or blender 28 with one or more pharmaceutically acceptable excipients, which the system 10 supplies through one or more feeders 30. The excipients are soluble in the liquid carrier and help stabilize the nanoparticles. The resulting slurry from the blender 28 enters a spray dryer 32, which drives off the liquid carrier and produces a dry granulation of nanoparticles and excipients.

Useful excipients include, without limitation, lactose, mannitol, sorbitol, sucrose, trehalose, xylitol, dextrates, dextran, dextrose, and the like. The amounts of any excipients added during granulation will depend on the desired drug loading in the dry granulation. In most cases, the API comprises from about 5% w/w to about 95% w/w of the dry granulation and often comprises from about 5% w/w to about 65% w/w of the dry granulation. For a discussion of useful excipients that may be used to stabilize the nanosuspension, see U.S. Pat. No. 5,571,536 to W. M. Eickhoff et al. and U.S. Pat. No. 6,153,225 to R. Lee & L. De Castro, which are herein incorporated by reference in their entirety and for all purposes.

Useful high-shear, wet granulators include, without limitation, twin-screw mixers, planetary mixers, high-speed mixers, extruder-spheronizers and the like. Other useful wet granulators include fluidized bed granulators. Like spray drying, fluidized bed granulation is a low-shear granulation method. However, as its name suggests, fluidized bed granulation involves spray-coating a fluidized bed of particles containing excipients (and optionally API), with a liquid suspension of API. In contrast, spray drying involves spraying an API slurry into a hot gas in order to produce granules; the slurry comprises discrete nanoparticles of API dispersed in a liquid carrier, as well as one or more excipients, which are dissolved in the liquid carrier. For a discussion of useful wet granulators, see M. Summers & M. Aulton, Dosage Form Design and Manufacture 25:364-78 (2d ed., 2001), the complete disclosure of which is herein incorporated by reference.

The resulting dry granulation (which has an average particle size of about 250 μm to about 2000 μm) may be stored, used to make drug product, or directly fed to an optional milling operation 34, where the size of the granulation is reduced to a median particle size of about 1 μm to about 80 μm. Useful milling equipment includes jet mills (dry), ball mills, hammer mills, and the like. The milled granulation is combined with additional pharmaceutically acceptable excipients, if necessary, from one or more solids feeders 36. The resulting mixture undergoes dry blending 38 (say, in a v-cone blender) to form a drug product, which may optionally undergo further operations, such as tableting or encapsulation 40, coating 42, and the like, to form the final dosage form of the drug product. For a discussion of drying, milling, dry blending, tableting, encapsulation, coating, and the like, see A. R. Gennaro (ed.), Remington: The Science and Practice of Pharmacy (20th ed., 2000); H. A. Lieberman et al. (ed.), Pharmaceutical Dosage Forms: Tablets, Vol. 1-3 (2d ed., 1990); and D. K. Parikh & C. K. Parikh, Handbook of Pharmaceutical Granulation Technology, Vol. 81 (1997), which are herein incorporated by reference.

For tablet dosage forms, depending on dose, the drug may comprise about 1% to about 80% of the dosage form, but more typically comprises about 5% to about 65% of the dosage form, based on weight. In addition to the drug substance, the tablets may include one or more disintegrants, surfactants, glidants, lubricants, binding agents, and diluents, either alone or in combination. Examples of disintegrants include, without limitation, sodium starch glycolate; carboxymethylcellulose, including its sodium and calcium salts; croscarmellose; crospovidone, including its sodium salt; PVP, methylcellulose; microcrystalline cellulose; one- to six-carbon alkyl-substituted HPC; starch; pregelatinized starch; sodium alginate; and mixtures thereof. The disintegrant will generally comprise about 1% to about 25% of the dosage form, or more typically, about 5% to about 20% of the dosage form, based on weight.

Tablets may optionally include surfactants, such as SLS and polysorbate 80; glidants, such as silicon dioxide and talc; and lubricants, such as magnesium stearate, calcium stearate, zinc stearate, sodium stearyl fumarate, sodium lauryl sulfate, and mixtures thereof. When present, surfactants may comprise about 0.2% to about 5% of the tablet; glidants may comprise about 0.2% to about 1% of the tablet; and lubricants may comprise about 0.25% to about 10%, or more typically, about 0.5% to about 3% of the tablet, based on weight.

As noted above, tablet formulations may include binders and diluents. Binders are generally used to impart cohesive qualities to the tablet formulation and typically comprise about 10% or more of the tablet based on weight. Examples of binders include, without limitation, microcrystalline cellulose, gelatin, sugars, polyethylene glycol, natural and synthetic gums, PVP, pregelatinized starch, HPC, and HPMC. One or more diluents may make up the balance of the tablet formulation. Examples of diluents include, without limitation, lactose monohydrate, spray-dried lactose monohydrate, anhydrous lactose, and the like; mannitol; xylitol; dextrose; sucrose; sorbitol; microcrystalline cellulose; starch; dibasic calcium phosphate dihydrate; and mixtures thereof.

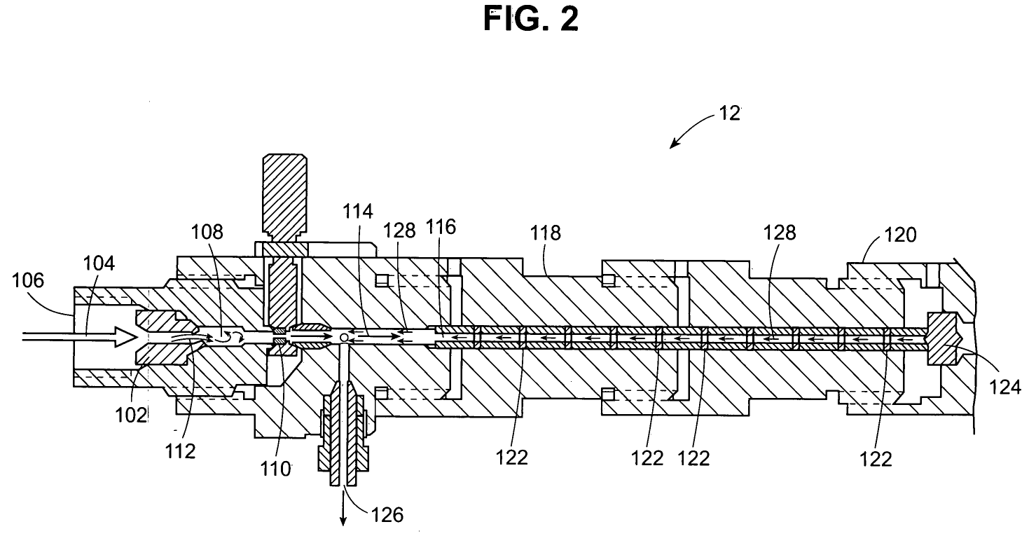

FIG. 2 shows a cross-sectional view of a modular high-pressure spray (jet) homogenizer 12, which is used to comminute the coarse dispersion into a nanoparticulate suspension or dispersion. The high-pressure spray homogenizer 12 includes a flow-coupling device 102, which directs the flow of the coarse dispersion of particles (represented by a first arrow 104) from a first port 106 into an expansion chamber 108, which is located immediately upstream of a nozzle 110. The expansion chamber 108 ensures that the flow is turbulent as it enters the nozzle 110. In other embodiments, a flow-coupling device (not shown) fills the expansion chamber 108 so that the flow of the coarse dispersion is laminar as it enters the nozzle 110. Turbulent flow upstream of the nozzle 110, which is represented by a second set of arrows 112, permits pre-mixing of the components of the coarse dispersion and increases cavitation, whereas laminar flow upstream of the nozzle 110 decreases cavitation.

The nozzle 110 converts the high pressure (up to 45,000 psig) coarse dispersion into a high velocity jet, which as shown by a third set of arrows 114 in FIG. 2, travels down a bore 116 formed by one or more process cells 118, a retaining cell 120, and washer-like, coaxial seals 122, which are sandwiched between adjacent process cells 118 or between a terminal process cell and the retaining cell 120. Upon reaching an end plug 124 located in the retaining cell 120, the flow reverses and returns down the bore 116, leaving the high-pressure spray homogenizer 12 via a second port 126. The primary jet flow 114 and the reverse (return) flow, which is indicated by a fourth set of arrows 128, comprise a countercurrent, core-annular flow that generates impact and shear forces that, along with cavitation, breakup (comminute) the solid particles.

In other embodiments, the end plug 124 may be removed. In one such embodiment, which is useful for comminuting a coarse dispersion of hard particles, the continuous (liquid) phase enters the high-pressure spray homogenizer 12 via the nozzle 110, while the coarse dispersion of hard particles enters the spray homogenizer 12 via a third port (not shown) that is adapted to receive the absent end plug 124. In this case, the primary jet flow is comprised of the continuous phase alone, while the “reverse” flow is comprised of the continuous phase and the coarse dispersion of hard particles.

In a parallel flow arrangement, which is useful for comminuting highly viscous, abrasive, or dry dispersions, the continuous (liquid) phase enters the high-pressure spray homogenizer 12 via the nozzle 110, while the viscous, abrasive, or dry dispersion enters the homogenizer 12 via the second port 126. The two streams interact downstream of the nozzle 110, forming a co-current, core-annular flow that exits the high-pressure spray homogenizer via the third port that is adapted to receive the absent end plug 124.

As noted above, impact, cavitation, and shear forces, as well as flow characteristics (turbulent or laminar) and process duration may be varied to accommodate different solid fracture characteristics of the API. For example, the size of the nozzle 110 can be changed to account for differences in viscosity among coarse dispersions and to control pressure, degree of cavitation, and flow rate, which may vary from about 225 mL/min to about 1800 mL/min. Since the process cells 118 absorb kinetic energy from the high velocity jet, the number of process cells 118 controls the duration and intensity of the comminuting process and along with the process cell geometry, influences the overall shear imparted. Thus, increasing the number of process cells decreases shear forces, while decreasing the number of process cells 118 increases particle impact forces, but decreases shear forces. Furthermore, utilizing a reverse flow configuration increases impact and shear forces, while a parallel flow arrangement decreases impact and shear forces. Also by selecting seals 122 having inner diameters (IDs) that are greater than the ID of the process cells 118 promotes turbulent flow, which increases impact forces. Likewise, selecting seals 122 having IDs that are the same as the ID of the process cells 118 results in less turbulent flow, thereby decreasing impact forces. For a detailed description of a useful high-pressure spray homogenizer 12, see U.S. Pat. No. 5,720,551 to T. Shechter; U.S. Pat. No. 6,443,610 to T. Shechter et al.; and U.S. Pat. No. 6,541,029 to R. Namba, the complete disclosures of which are herein incorporated by reference.

The disclosed method may be used to prepare pharmaceutical nanoparticle suspensions or dispersions, granulations, and final dosage forms comprised of any active pharmaceutical ingredient. Useful APIs include those that belong to a variety of known classes of drugs including, for example and without limitation, analgesics, anti-inflammatory agents (including NSAIDs), anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators, xanthines, and antiviral agents.

Particularly useful drug substances or active pharmaceutical ingredients include those intended for oral administration or parenteral administration, including intravenous and intramuscular administration. A description of these classes of drugs and a listing of species within each class can be found in Martindale, The Extra Pharmacopoeia (29th ed. 1989), which is hereby incorporated by reference. The drug substances are commercially available or can be prepared by known techniques.

Useful NSAIDs include those described in U.S. Pat. No. 5,552,160 to Liversidge et al., and include acidic compounds and nonacidic compounds. Useful nonacidic NSAIDs include, without limitation, nabumetone, tiaramide, proquazone, bufexamac, flumizole, epirazole, tinoridine, timegadine, and dapsone, as well as COX-2 selective inhibitors, such as rofecoxib, celecoxib, and valdecoxib. Useful carboxylic acid NSAIDs include, without limitation, salicylic acids and esters thereof, such as aspirin; phenylacetic acids such as diclofenac, alclofenac, and fenclofenac; carbo- and heterocyclic acetic acids such as etodolac, indomethacin, sulindac, tolmetin, fentiazac, and tilomisole; propionic acids such as carprofen, fenbufen, flurbiprofen, ketoprofen, oxaprozin, suprofen, tiaprofenic acid, ibuprofen, naproxen, fenoprofen, indoprofen, and pirprofen; and fenamic acids such as flufenamic, mefenamic, meclofenamic, and niflumic. Suitable enolic acid NSAIDs include, without limitation, pyrazolones such as oxyphenbutazone, phenylbutazone, apazone, and feprazone; and oxicams such as piroxicam, sudoxicam, isoxicam, and tenoxicam.

Useful anticancer agents include those described in U.S. Pat. No. 5,399,363 to Liversidge et al., which include, without limitation, alkylating agents, antimetabolites, natural products, hormones and antagonists, and miscellaneous agents, such as radiosensitizers. Examples of alkylating agents include, without limitation, alkylating agents having the bis-(2-chloroethyl)-amine group such as chlormethine, chlorambucile, melphalan, uramustine, mannomustine, extramustinephoshate, mechlore-thaminoxide, cyclophosphamide, ifosfamide, and trifosfamide; alkylating agents having a substituted aziridine group such as tretamine, thiotepa, triaziquone, and mitomycine; alkylating agents of the alkyl sulfonate type, such as busulfan, piposulfan, and piposulfam; alkylating N-alkyl-N-nitrosourea derivatives, such as carnustine, lomustine, semustine, or streptozotocine; and alkylating agents of the mitobronitole, dacarbazine, and procarbazine type.

Examples of antimetabolites include, without limitation, folic acid analogs, such as methotrexate; pyrimidine analogs such as fluorouracil, floxuridine, tegafur, cytarabine, idoxuridine, and flucytosine; and purine derivatives such as mercaptopurine, thioguanine, azathioprine, tiamiprine, vidarabine, pentostatin, and puromycine. Examples of natural products include vinca alkaloids, such as vinblastine and vincristine; epipodophylotoxins, such as etoposide and teniposide; antibiotics, such as adriamycine, daunomycine, doctinomycin, daunorubicin, doxorubicin, mithramycin, bleomycin, and mitomycin; enzymes, such as L-asparaginase; biological response modifiers, such as alpha-interferon; camptothecin; taxol; and retinoids, such as retinoic acid.

Examples of hormones and antagonists include, without limitation, adrenocorticosteroids, such as prednisone; progestins, such as hydroxyprogesterone caproate, medroxyprogesterone acetate, and megestrol acetate; estrogens, such as diethylstilbestrol and ethinyl estradiol; antiestrogens, such as tamoxifen; androgens, such as testosterone propionate and fluoxymesterone; antiandrogens, such as flutamide; and gonadotropin-releasing hormone analogs, such as leuprolide.

Examples of miscellaneous agents include, without limitation, radiosensitizers, such as, for example, 1,2,4-benzotriazin-3-amine 1,4-dioxide (SR 4889) and 1,2,4-benzotriazine-7-amine 1,4-dioxide (WIN 59075); platinum coordination complexes such as cisplatin and carboplatin; anthracenediones, such as mitoxantrone; substituted ureas, such as hydroxyurea; and adrenocortical suppressants, such as mitotane and aminoglutethimide. In addition, the anticancer agent can be an immunosuppressive drug, such as cyclosporine, azathioprine, sulfasalazine, methoxsalen, and thalidomide

The disclosed method is useful for preparing pharmaceutical nanoparticle suspensions or dispersions, granulations, and final dosage forms containing an API that is poorly water-soluble. Furthermore, the disclosed method is particularly useful for preparing pharmaceutical nanoparticle suspensions or dispersions, granulations, and final dosage forms comprised of an API having a dose to aqueous solubility ratio greater than about 100 at a pH of about 5 to about 7.

### EXAMPLES

The following examples are intended to be illustrative and non-limiting, and represent specific embodiments of the present invention.

Although number distribution is typically used to express particle size, it can be misleading when there are larger particles present in the distribution. The number distribution is normally lower than the volume distribution. However, since pharmaceutical dosing is based on mass, volume distribution is a more accurate measure of the particle size distribution since a small percentage of larger particles can account for a considerably higher percentage of the total weight of the particles. Hence, unless stated otherwise, volume distribution is used throughout the specification to report particle size distribution.

Materials

CPD-1 (API having a melting point of 176-178° C.), celecoxib (API having a melting point of 160-163° C.), naproxen, USP Water, SLS, TWEEN 80, AVICEL PH 101, FAST FLO Lactose, CAB-O-SIL (fumed silica), magnesium stearate, PVP K-30, and croscarmellose sodium.

Equipment & Instruments

DeBee 2000 (high-pressure spray homogenizer, Model 2510), IKA T25 B S1, Silverson L4R Mixer, Tekmar Mixer, Haake Twin Screw Mixer, Ivek Pump (Model Number 102144-2), K Tron Feeder, Coulter LS 230 Particle Size Analyzer, CPS Instruments, Inc Disc Centrifuge Model DC18000, Brookhaven 90 Plus Particle Size Analyzer, Agilent UV-visible spectrophotometer HP8453, CTechnologies IO Fiber Optic Dissolution System with VanKel VK7010 dissolution bath, Quadro Comil, V Blender, Turbula T2 Mixer, Strea Fluid Bed Dryer, Presstor, Computrac Moisture Analyzer, Erweka Disintegration Tester (Model No. 51939).

Methods

A liter of coarse suspension (solids concentration 1-80% (w/w), surfactant 0-1% (w/w)) was processed using various cell configurations (operating pressure range 2K-45K psig, backpressure 0-5K psig) (TABLE 2). The nanosuspension formed was granulated with excipients using a Twin-Screw Mixer (to stabilize the nanoparticles), which was then dried, milled, blended and tableted.

Coarse Suspension Formation and High Pressure Processing

A predetermined quantity of a surfactant was dissolved in appropriate quantity of USP water by gentle stirring to prevent foaming. The surfactant solution was then poured in a 1 L stainless steel vessel containing the drug substance. Vigorous mixing was performed to wet and uniformly suspend the coarse suspension using a Silverson mixer. The suspension was then transferred into a reservoir of the high-pressure spray homogenizer. An IKA rotor-stator mixer was installed in the reservoir to prevent settling during processing, which allowed for a surprisingly high concentration of solids to be processed. A much lower solids concentration can be processed if such a mixer is not employed. See WO 03/045353A1. Unlike the Avestin B3 (piston gap homogenizer) which utilizes horizontal flow and thereby necessitates the use of vertical flow (e.g., Gaulin APV homogenizers), no blocking of the nozzle using the modular high-pressure spray homogenizer was observed for concentrations as high as 80% w/w. TABLE 2 lists different cell geometries and processing conditions used to prepare the suspensions. A heat exchanger coupled to a chiller was employed to maintain the process temperature.

Twin Screw Continuous Mixer Granulation, Drying, Milling and Blending

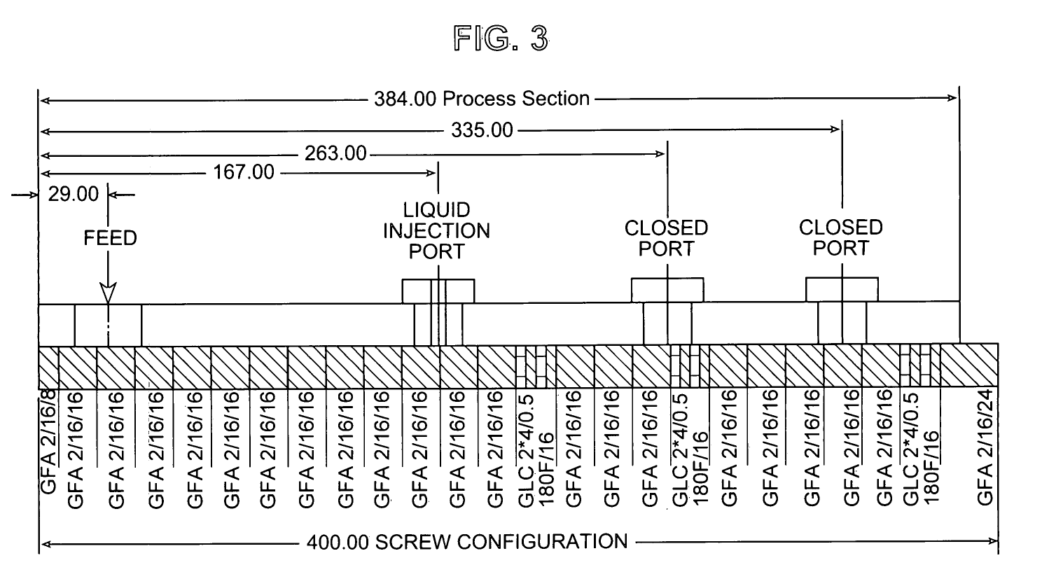

FIG. 3 shows the screw design of the Haake twin-screw mixer (TSM), which was used to uniformly disperse and separate the nanosuspension on to suitable excipients. The TSM is a continuous process and imparts mixing and shearing, which can uniformly disperse and separate nanoparticles and hence prevent agglomeration and crystal growth, thereby forming a solid state stabilized nanomaterial. An Ivek dual head piston pump was used to consistently feed the suspension and K-Tron loss-in-weight feeder was employed to feed the excipient or excipients into the TSM. TABLE 3 lists tablet formulations.

Tableting

Presster Compaction Replicator (simulating Betapress 16 station, turret speed-50 rpm) was employed to make tablets at 5, 10, 15, and 20 kP hardness. For 500 mg CPD-1 tablets, a 12/32 inch flat faced round and for 750-mg Naproxen tablets 0.748×0.426×0.045 inch oval concave tooling was employed.

Results

Particle Size

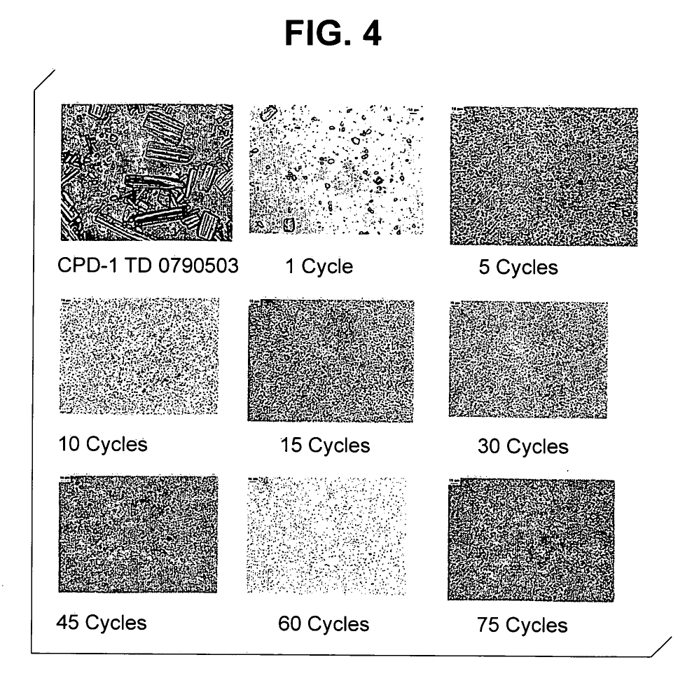

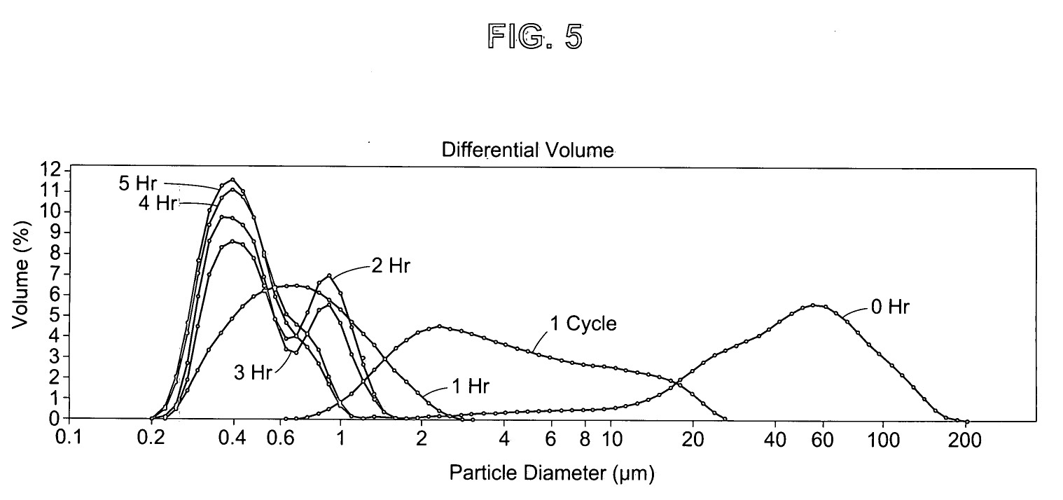

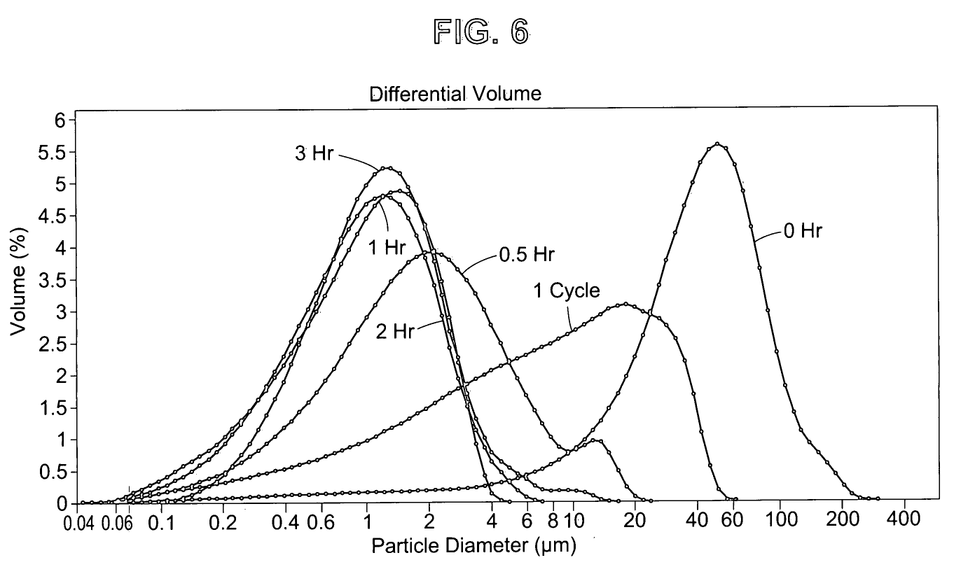

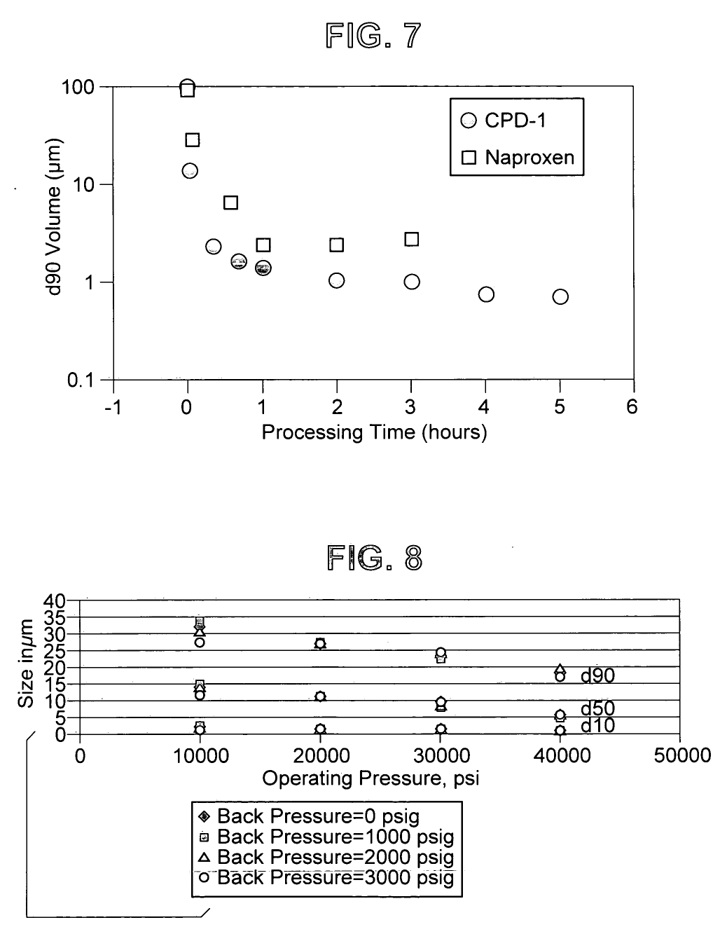

TABLE 4 and FIG. 4 to FIG. 7 show the effect of the number of cycles on particle size. The mean particle size and distribution decreased as the processing time was increased. A higher initial particle size of the coarse suspension required longer processing time to form nanoparticles. One observation was that the first pass typically reduced the particle size considerably and the size distribution was also narrower compared to the coarse suspension. The overall process duration is much shorter compared to the ball mill technique, i.e., a few hours versus several days. Though not bound to any particular theory, this may be attributed to enhanced particle-particle interaction (shear, impact and attrition) which can be controlled and modulated to suit the drug substance characteristics.

Effect of Operating and Backpressure

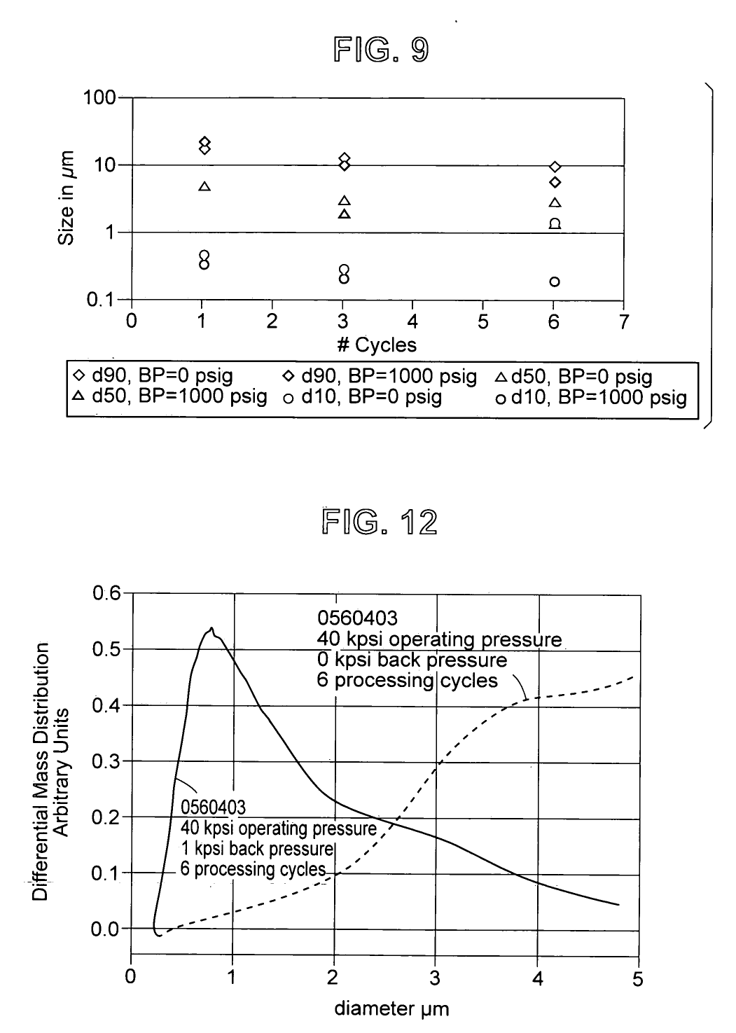

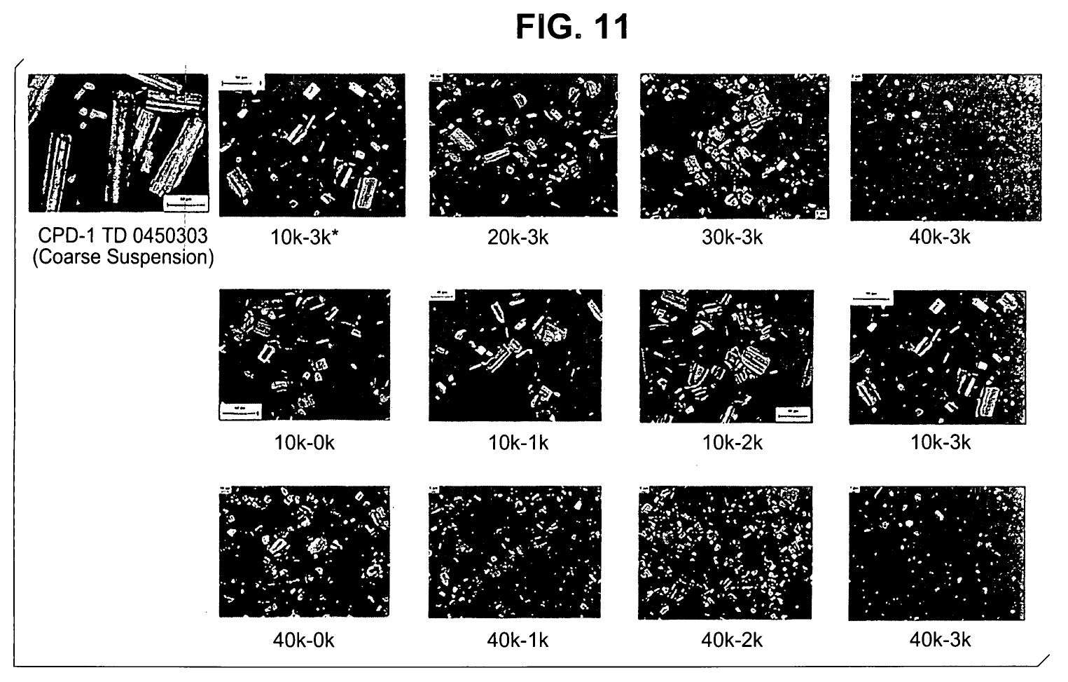

TABLE 5 and FIG. 8 to FIG. 12 show the effects of operating pressure on the size reduction of CPD-1. As can be seen in the figures, increasing the operating pressure up to 45,000-psig results in significant reduction in the particle size of CPD-1. Also, these results indicate that operating pressure has a greater effect on the larger particles (d90 values) compared to the smaller particles (d10 values).

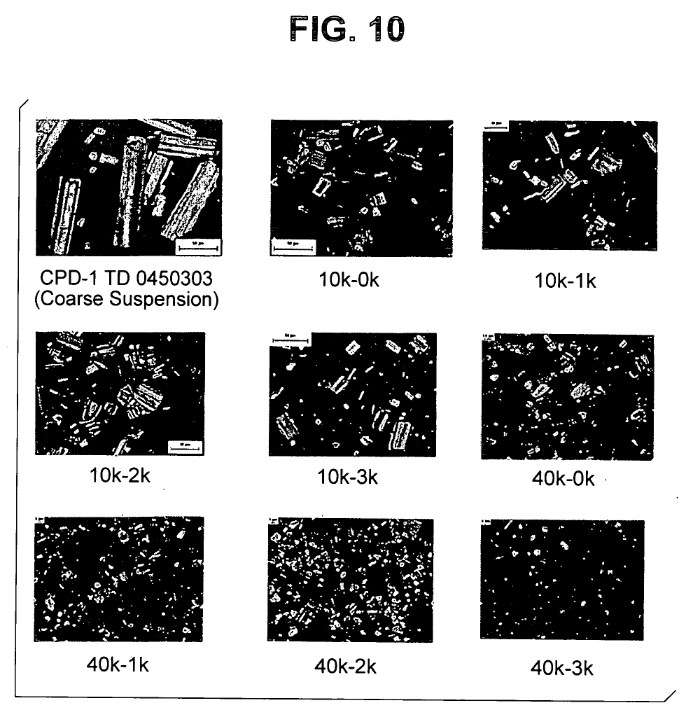

Backpressure, on the other hand, has a less dramatic effect on the particle size reduction. FIG. 10 illustrates the combined effect of operating and backpressures on the dynamics of the expanding fluid. Although the differential pressure (operating pressure minus back pressure) has a direct effect on the kinetic energy imparted to the expanding fluid, it also controls the residence time of the fluid within the process cells. The relative contribution of each of these mechanisms dictates the final particle size. From the results shown in FIG. 10 to FIG. 12, it appears that the former mechanism was prominent at higher operating pressures, while the latter seemed to control the behavior at lower operating pressures. While this holds true for 1 cycle of processing, higher backpressures appear to cause significant particle size reduction when multiple processing cycles are involved. In summary, higher values of both operating and backpressure are conducive to forming submicron to nanoparticles by multiple cycle processing. This pressure control also affects the levels of shear, impact and cavitation experienced by the particles.

As shown in FIG. 12, differential API mass distributions of TD0560403 indicate that a setting of 1000 (1K) psig backpressure is more effective for size reduction compared to a setting of zero (0) psig. The distributions are area normalized and only the small diameter portion of the zero backpressure mass distribution is resolved. Increasing the backpressure increased process duration per cycle and particle-particle interactions and resulted in lower and narrower particle size distribution. Typical piston gap arrangements have no control over the backpressure.

Effect of Concentration

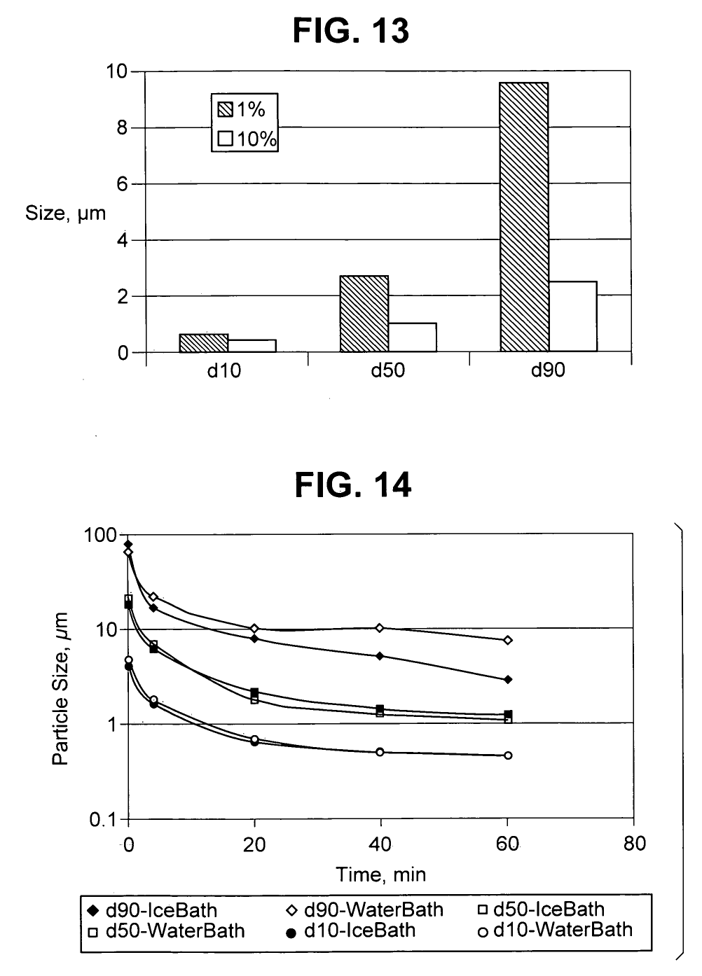

TABLE 6 and FIG. 13 show the effects on particle size distribution of the concentration of CPD-1 in the dispersion at two levels of surfactants. As can be seen in FIG. 13, the particle size decreases with increasing concentration. Though not bound to any particular theory, it appears that the concentration of solids in the material being processed dictates the final particle size through two competing mechanisms. An increase in concentration translates into an increase in the particle-particle attrition within the process cells. Alternatively, increased solid concentration also means an increase in the drag of the fluid (viscosity) that impedes the achievable kinetic velocities. Particle attrition as a function of the surface tension of the fluid (cavitation) may also play a role. For simplicity, it is treated independent of the solid concentration.

Effect of Temperature

FIG. 14 shows the effect of temperature on the particle size of CPD-1 suspensions. As indicated in FIG. 14, no significant differences can be seen in the d10 and d50 values of the suspensions processed at different temperatures. On the other hand, the d90 value of the material processed at 15° C. is significantly less compared to that at 30° C. Given that the larger particle sizes influence the d90 value, the behavior seen in FIG. 14 can be attributed to particle agglomeration at higher temperature. Temperature of the product thus has multiple implications in the manner it is processed by a size reduction system. Though not bound to any particular theory, the primary effects of temperature on the process ability of suspensions are mediated through alterations in such properties as viscosity, surface tension, kinetic energy, particle hardness, etc. Secondarily, temperature also influences the tendency of the particles to agglomerate and fuse. The secondary effects are more prominent in processes where multiple cycles are involved. Such effects are evident in CPD-1 suspensions where the temperature control was tested using two different sinks. The product temperature when ice and water baths were used as sinks was, respectively, less than 15° C. and 30° C. Further reduction in temperature during processing is expected to not only prevent agglomeration but also make the drug substance more brittle and hence reduce the overall process time. The most effective coolant temperature is well below room temperature.

Effect of Surfactant Type and Concentration

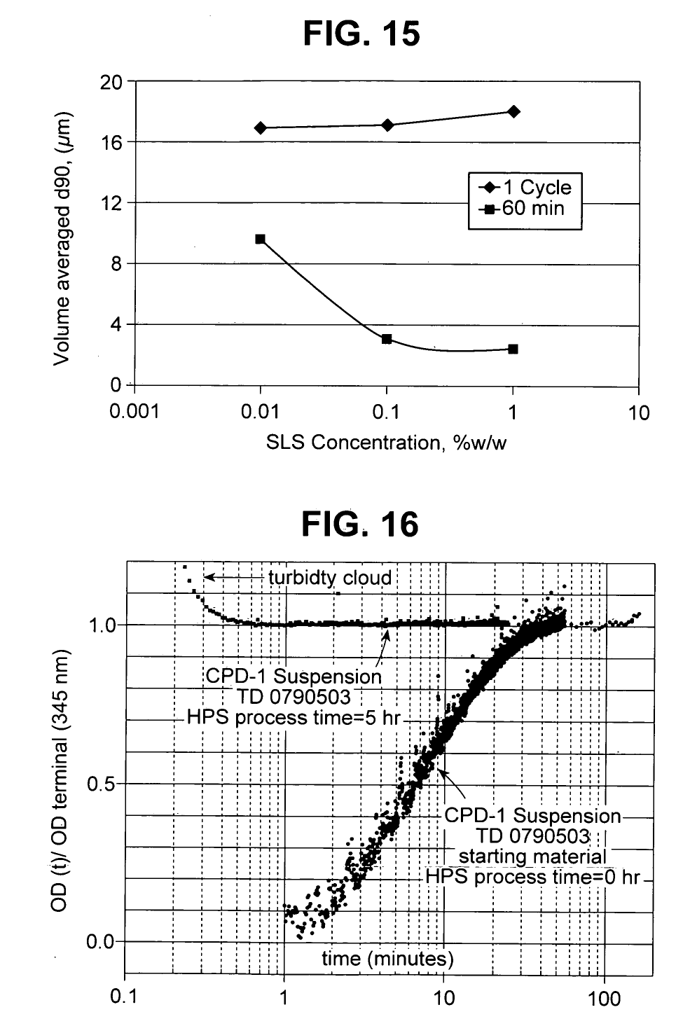

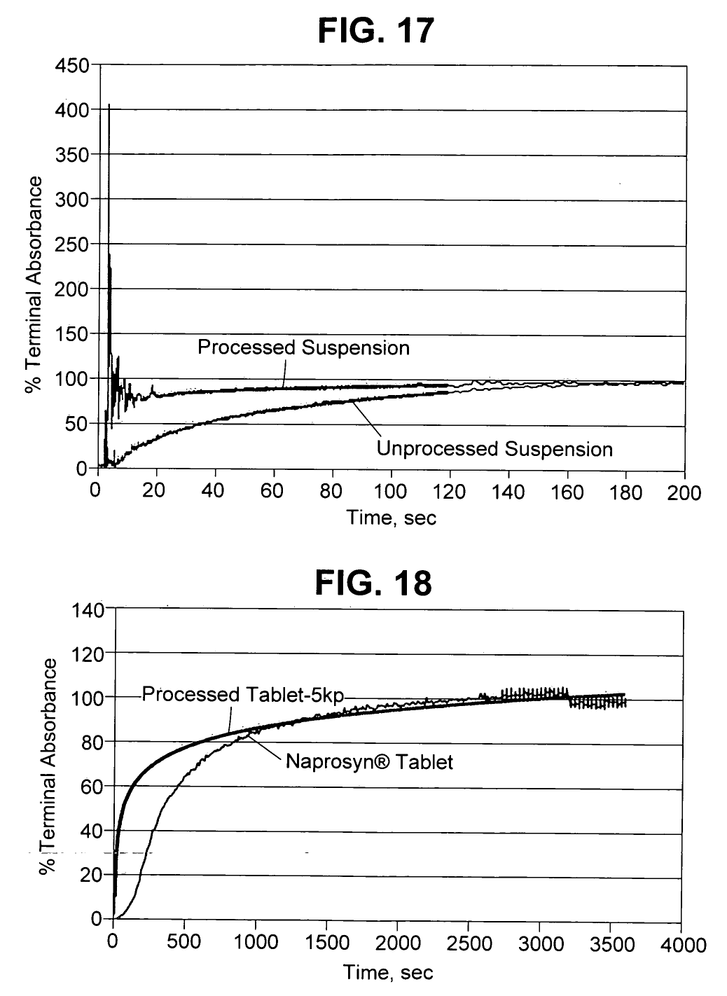

TABLE 7 and FIG. 15 show the effects of surfactant concentration on particle size. Though not bound to any particularly theory, the surfactant appears to influence the particle size during processing by affecting the surface tension of the continuous phase. As can be seen in FIG. 17, reduced surface tension at higher levels of SLS in the suspension had a slightly negative effect on the initial particle size of CPD-1 (1 cycle). However, once particle size reduction occurs, it appears that a higher level of surfactant is required to stabilize the particles. This is evident from TABLE 7, where higher level of surfactant leads to reduced agglomeration.

Dissolution Kinetics of Suspension

To determine the dissolution kinetics of the starting material suspension (coarse dispersion) and the processed suspension, studies were performed on TD0790503 suspension (10% CPD-1, 1% Tween 80). The starting material suspension was compared to the five (5) hour processing time suspension. The d90 of the starting material was 90 μm and the d90 of the suspension after five hours of processing was 0.9 μm. A dose of 100 mg API/900 mL dissolution medium was tested.

A VanKel VK7010 Type II (paddles) with interfaced IO fiber optic dissolution system was used at paddle speed=150 rpm (since starting material settles at lower rpm). The following data acquisition parameters were used: record optical density of dissolution medium at 345 nm with in situ fiber optic dip probe, path length =2×0.5 cm=1 cm, data sampling rate=1 Hz for first hour, 0.003 Hz for subsequent 4 hours.

FIG. 16 shows the dissolution kinetics for CPD-1 suspension TD0790503, which compares the starting material suspension to the five (5) hour processing time suspension. The solid circles labeled CPD-1 suspension TD0790503, process time=5 hours, reveal the solvation kinetics of the nanosuspension (d90=0.9 μm). The solid circles labeled CPD-1 suspension TD0790503, starting material process time =0 hours, reveal the solvation kinetics of the unprocessed suspension (d90=90 μm). Due to low initial scattering, the optical density of the unprocessed suspension is negligible at early times (t<1 minute), and then increases via absorption as the CPD-1 solvates. The time required to attain 90% of the final value (i.e., 90 mg of the 100-mg API dose) is 20 minutes. Because of the high initial scattering, the optical density of the nanosuspension TD0790503, process time=5 hours, is not negligible (the number density of the nanosuspension is 106-fold greater than that of the starting material). The recorded optical density decreases as the nanoparticulate CPD-1 solvates. The time required for the nanosuspension to attain the terminal optical density value is<1 minute.

Dissolution testing of naproxen suspensions was performed in type-Il dissolution apparatus (Distek) employing online fiber optic dissolution probes (CTechnologies). The conditions for dissolution testing of naproxen suspensions included: 900 mL of 1% Tween 80 in water as dissolution medium that was maintained at 37° C. and a paddle speed of 50 rpm. Utilizing fiber optic probes with a path length of 1 cm (2×0.5 cm), the absorbance from naproxen was recorded at 332 nm. Data collection was performed every 0.5 seconds for the first 2 minutes and at 1 Hz subsequently.

Naproxen samples tested included unprocessed naproxen suspended in water using 1% Tween 80 and the same processed by the modular high-pressure spray homogenizer for 5 hours at an operating and backpressures of 45000 and 3000 psig, respectively. The d90 values of the unprocessed and processed naproxen suspensions were 23.68 μm and 2.8 μm, respectively. A 100 mg of these suspensions (40 mg naproxen) were delivered to dissolution vessels containing 900 mL of 1% Tween 80 medium.

FIG. 17 shows the dissolution profiles of naproxen suspensions. The processed naproxen suspension behaved in a similar manner compared to the processed CPD-1 suspension. As shown in FIG. 23, there is a large initial surge in the optical density upon introduction of the processed suspension to the dissolution media. This is expected to originate from the absorbance by naproxen and from the scattering by nanoparticles. As the nanoparticles start to dissolve, the effect of scattering decreases until the final absorbance reaches an asymptotic value. Such behavior is not seen in the unprocessed suspension because of the absence of nanoparticles. The t80 values (time at which 80% of dose dissolved) for processed and unprocessed suspensions were estimated from FIG. 17 and were, respectively, about 12 seconds and 104 seconds. A nine-fold enhancement in the dissolution rate was therefore evident when the naproxen particles were reduced in size 10-fold.

Tablet Disintegration and Dissolution

TABLE 8 shows the target tablet harness and disintegration time data. Both CPD-1 (TD0820603 and 0870703) and Naproxen (TD090703 and 0910803; TD0980803 and 0990803) tablets were made on Presster Compaction Simulator. For CPD-1, the target tablet weight was 500 mg (equivalent to 100 mg dose) and for naproxen the target tablet weight was 750 mg (equivalent to 250 mg dose). Compression force vs. hardness profiles were generated for 5, 10, 15, and 20 kP tablet hardness. High suspension concentrations allow for high-shear wet granulation rather then employing fluid-bed or spray drying processes.

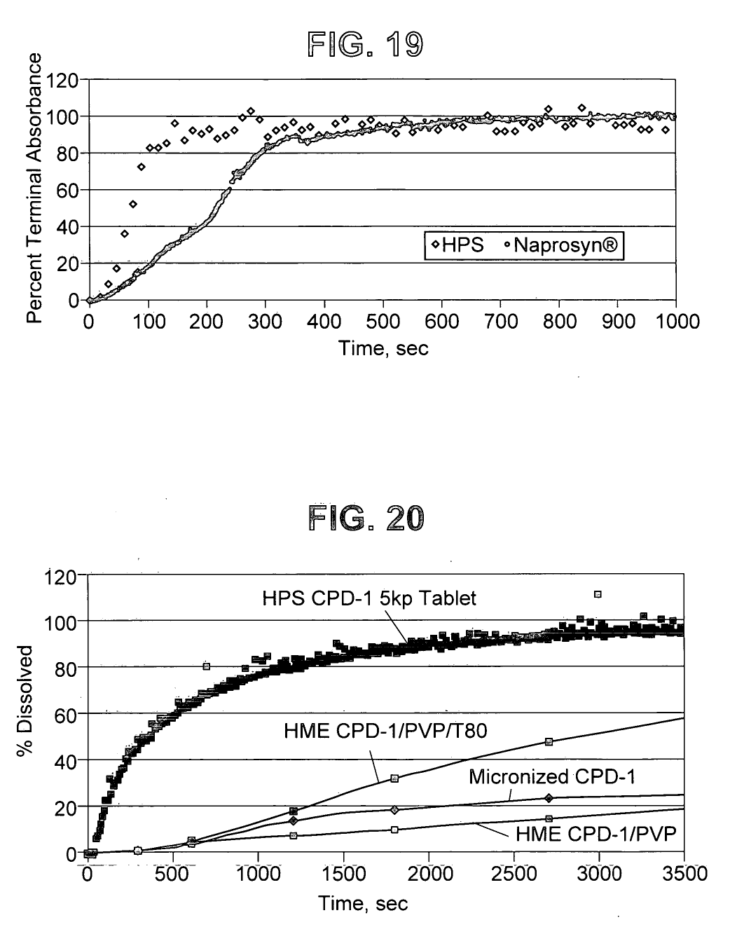

FIG. 18 and FIG. 19 show dissolution profiles of nanoparticulate naproxen tablets versus commercially available Naprosyn® tablets in dissolution media at two different pH, and indicate a faster dissolution rate for the nanoparticulate naproxen.

FIG. 20 compares the dissolution profile of nanoparticulate CPD-1 to micronized CPD-1 and solid dispersions of CPD-1 in PVP, which were obtained by hot-melt extrusion. The dissolution profile of nanoparticulate CPD-1 tablets shows enhanced dissolution profile compared to the hot-melt process and micronized drug substance.

Celecoxib Nanoparticulate Dispersion

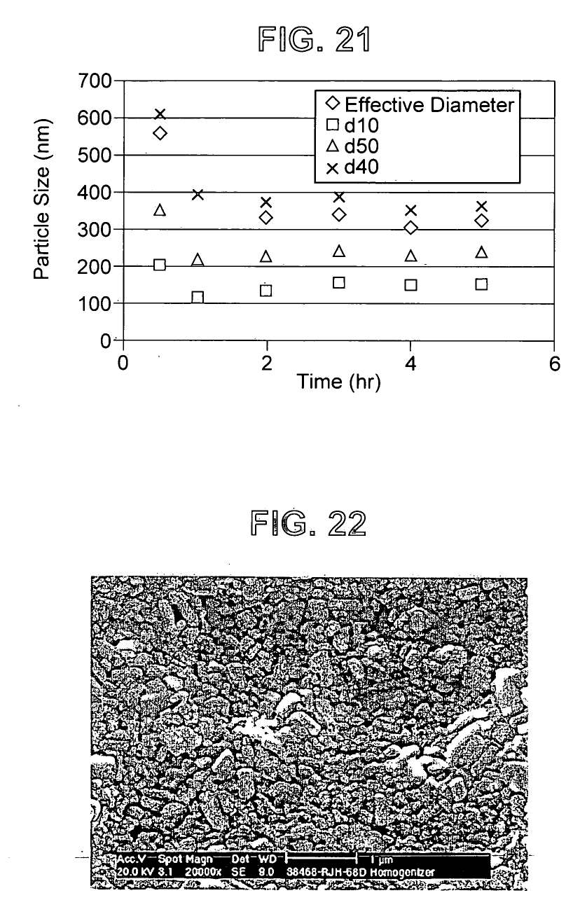

FIG. 21 and FIG. 22 provide data for a CPD-2 dispersion prepared using the high-pressure spray homogenizer. TABLE 9 lists different cell geometries and processing conditions used to prepare the celecoxib suspensions using the HPS homogenizer. FIG. 22 shows d10 , d50, d90 and effective diameter based on volume of the celecoxib dispersion as a function of process time. The data were obtained using photon correlation spectrophotometer. FIG. 22 is a scanning electron photomicrograph of celecoxib nanoparticle dispersion.

It should be noted that, as used in this specification and the appended claims, singular articles such as “a,” “an,” and “the,” may refer to a single object or to a plurality of objects unless the context clearly indicates otherwise. For example, reference to a composition containing “a compound” may include a single compound or two or more compounds. In addition, the above description is intended to be illustrative and not restrictive. Many embodiments will be apparent to those of skill in the art upon reading the above description. The scope of the invention should, therefore, be determined with reference to the appended claims, along with the full scope of equivalents to which such claims are entitled. The disclosures of all articles and references, including patents, patent applications and publications, are herein incorporated by reference in their entirety and for all purposes.

TABLE 2
Experimental Conditions (CPD-1 and Naproxen)

| | | | | | | | | | Operating Conditions | | |
|---|---|---|---|---|---|---|---|---|---|---|---|
| | | | | | | Cell | Seal | Nozzle | Operating | Back | |
| | | | | | | Diam- | Diam- | Diam- | Pressure | Pressure | |
| Exp. | | Drug Substance | Surfactant | Cell | No. of | eter | eter | eter | (1000 | (1000 | Cycles(C)/ |
| No. | Lot No. | (% w/w) | (% w/w) | Set-Up^{a,b} | Cells | (mm) | (mm) | (mm) | psig) | psig) | Duration (min or hr)^{c} |
| 1 | 0410303 | 10% Naproxen | 1% SLS | RF | 11 | 0.50 | 0.50 | 0.10 | 45 | 1, 2.5, 4 | 5C/45 min |
| | 0420303 | (Lot 32K1300) | | | | | | | | | |
| 2 | 0450303 | 1% CPD-1 | 1% SLS | RF | 11 | 0.50 | 0.50 | 0.10 | 10, 20, 30, | 0 1, 2, 3 | 1C |
| | | | | | | | | | 40, 45 | | |
| 3 | 0560403 | 1% CPD-1 | 1% SLS | RF | 11 | 0.50 | 0.50 | 0.13 | 40 | 0 | 1C, 3C, 6C |
| 4 | 0640403 | 1.25% CPD-1 | 0.1% SLS | RF | 11 | 0.50 | 0.50 | 0.13 | 45 | 3 | 24 min |
| 5 | 0680503 | 1% CPD-1 | 0.01% SLS | RF | 11 | 0.50 | 0.50 | 0.10 | 45 | 3 | 1C, 20, 40, 60 min |
| 6 | 0690503 | 1% CPD-1 | 0.1% SLS | RF | 11 | 0.50 | 0.50 | 0.10 | 45 | 3 | 1C, 20, 40, 60 min |
| 7 | 0700503 | 1% CPD-1 | 1% SLS | RF | 11 | 0.50 | 0.50 | 0.10 | 45 | 3 | 1C, 2 C |
| | 0710503 | 10% CPD-1 | 0.01% SLS | | | | | | | | 20, 40, 60 min |
| 8 | 0720503 | 10% CPD-1 | 0.1% SLS | RF | 11 | 0.50 | 0.50 | 0.10 | 45 | 3 | 20, 40, 60 min |
| 9 | 0730503 | 10% CPD-1 | 1% SLS | RF | 11 | 0.50 | 0.50 | 0.10 | 45 | 3 | 40, 60 min |
| 10 | 0790503 | 10% CPD-1 | 1% Tween 80 | RF | 11 | 0.50 | 0.50 | 0.13 | 45 | 3 | 20 min, 2, 3, 4, 5 hr |
| 11 | 0800603 | 10% Naproxen | 0.1 SLS | RF | 11 | 0.50 | 0.50 | 0.10 | 45 | 3 | 4 hr |
| | | (Lot 072K1806) | | | | | | | | | |
| 12 | 0810603 | 58% CPD-1 | 1% Tween 80 | RF | 11 | 0.50 | 0.50 | 0.10 | 45 | 3 | 1C, 20 min |
| 13 | 0820603 | 40% CPD-1 | 1% Tween 80 | RF | 11 | 0.50 | 0.50 | 0.10 | 45 | 3 | 3 hr |
| 14 | 0900703 | 40% Naproxen | 1% Tween 80 | RF | 6 | 0.50 | 0.50 | 0.13 | 45 | 3 | 3 hr |
| | | Lot GG01 | | | | | | | | | |
| 15 | 0980803 | 40% Naproxen | 1% Tween 80 | RF | 11 | 0.50 | 0.50 | 0.13 | 45 | 3 | 5 hr |
| | | Lot 22704HB | | | | | | | | | |

Note
^{a}Turbulent coupling employed for all experiments;
^{b}RF: Reverse Flow Set-Up;
^{c}1 cycle = 4 minutes; 15 cycles = 1 hour; 30 cycles = 2 hours; 45 cycles = 3 hours; 60 cycles = 4 hours; 75 cycles = 5 hours;
^{d}For CPD-1 Lot XH210601 was used for all studies

TABLE 3
Tablet Formulations

| | | | | | Initial | | Tablet |
|---|---|---|---|---|---|---|---|
| | | Ivek Pump | Feeder Rate | | Moisture | Final Moisture | Weight |
| Lot No. | Tablet Formulation | Rate (g/min) | (g/min) | Screw RPM | (%) | (%) | (mg) |
| TD 0820603 | 20.0% CPD-1 | 17 | 24 | 150 | 28.4 | 3.9 | 500 |
| | 0.5% Tween 80 | | | | | | |
| | 1.0% Povidone | | | | | | |
| | 67.5% Avicel PH 101 | | | | | | |
| | 5.0% Fast Flo Lactose | | | | | | |
| | 5.0% Explotab | | | | | | |
| | 0.5% Cab O Sil | | | | | | |
| | 0.5% Magnesium Stearate | | | | | | |
| TD 0900703 | 34.6% Naproxen | 17 | 24 | 150 | 37.4 | 2.0 | 750 |
| | 1.0% Povidone | | | | | | |
| | 0.88% Tween 80 | | | | | | |
| | 57.52% Avicel PH 101 | | | | | | |
| | 5.0% Ac Di Sol | | | | | | |
| | 0.5% Cab O Sil | | | | | | |
| | 0.5% Magnesium Stearate | | | | | | |
| TD 0980803 | 34.6% Naproxen | 17 | 24 | 156 | 25.5 | 1.7 | 750 |
| | 1.0% Povidone | | | | | | |
| | 0.88% Tween 80 | | | | | | |
| | 57.52% Avicel PH 101 | | | | | | |
| | 5.0% Ac Di Sol | | | | | | |
| | 0.5% Cab O Sil | | | | | | |
| | 0.5% Magnesium Stearate | | | | | | |

TABLE 4
Effect of Number of Cycles

| | Operating Conditions | | | | | | | | |
|---|---|---|---|---|---|---|---|---|---|
| | Operating | Back- | Cycles (c) | Laser Diffraction^{¥} | | | Laser Diffraction^{φ} | | |
| | Pressure | pressure | Duration | (μm) | | | (μm) | | |
| Lot No. | (psig) | (psig) | (min/hr) | d10 | d50 | d90 | d10 | d50 | d90 |
| 0800603 | 45K | 3K | 0 min | 6.4 | 21.7 | 46.8 | 2.09 | 3.23 | 7.18 |
| | | | 1 c | NA^{ζ} | NA | NA | NA | NA | NA |
| | | | 4 hr | 0.43 | 1.04 | 2.35 | 0.22 | 0.34 | 0.70 |
| 0790503 | 45K | 3K | 0 min | 14.94 | 45.79 | 97.63 | 1.80 | 2.68 | 5.60 |
| | | | 1 c | 1.53 | 3.78 | 13.48 | 0.91 | 1.37 | 2.42 |
| | | | 1 hr | 0.36 | 0.68 | 1.38 | 0.27 | 0.37 | 0.64 |
| | | | 2 hr | 0.33 | 0.52 | 1.03 | 0.28 | 0.36 | 0.53 |
| | | | 3 hr | 0.31 | 0.47 | 0.97 | 0.27 | 0.35 | 0.49 |
| | | | 4 hr | 0.30 | 0.43 | 0.73 | 0.26 | 0.34 | 0.48 |
| | | | 5 hr | 0.29 | 0.42 | 0.70 | 0.26 | 0.33 | 0.47 |
| 0900703 | 45K | 3K | 0 min | 10.21 | 41.54 | 90.79 | — | — | — |
| | | | 1 c | 0.79 | 7.61 | 28.30 | | | |
| | | | 3 hr | 0.42 | 1.13 | 2.68 | | | |

^{¥}Stated values are for volume distributions
^{φ}Stated values are for number distribution
^{‡}Large diameter fraction of ensemble not completely resolved, therefore stated values are lower limits
^{ζ}NA = Not available.

TABLE 5
Effect of Operating and Backpressure

| | Operating Conditions | | | | | |
|---|---|---|---|---|---|---|
| | Operating | Backpressure | Cycles (C)/Duration | LD | | |
| Lot No. | Pressure (psig) | (psig) | (min or hr) | d10 | d50 | d90 |
| TD 0450303 | 10K | 0K | 1C | 2.02 | 14.14 | 31.85 |
| | | 1K | | 2.06 | 14.77 | 33.65 |
| | | 2K | | 1.78 | 13.32 | 30.79 |
| | | 3K | | 1.38 | 11.42 | 27.56 |
| | 20K | 0K | 1C | 1.51 | 11.18 | 26.68 |
| | | 1K | | 1.14 | 11.14 | 27.19 |
| | | 2K | | 1.33 | 11.10 | 27.28 |
| | | 3K | | 1.30 | 10.95 | 26.82 |
| | 30K | 0K | 1C | 1.17 | 8.31 | 22.62 |
| | | 1K | | 1.13 | 8.45 | 23.70 |
| | | 2K | | 1.11 | 8.70 | 24.49 |
| | | 3K | | 1.78 | 9.59 | 24.28 |
| | 40K | 0K | 1C | 0.82 | 5.88 | 17.46 |
| | | 1K | | 0.77 | 4.93 | 17.86 |
| | | 2K | | 0.77 | 6.09 | 19.94 |
| | | 3K | | 0.79 | 5.57 | 17.24 |

TABLE 6
Effect of Concentration Data Shown for Processing Time = 1 hour

| | Operating Conditions | | | LD | | |
|---|---|---|---|---|---|---|
| | Operating | Back Pressure | Cycles (C)/Duration | (μm) | | |
| Lot No. | Pressure (1000 psig) | (1000 psig) | (min/hr) | d10 | d50 | d90 |
| TD 0680503 | 45 | 3 | 0 | 4.02 | 19.29 | 59.6 |
| (1% CPD-1) | | | 60 min | 0.42 | 0.95 | 2.22 |
| TD 0710503 | 45 | 3 | 0 | 14.94 | 45.79 | 97.63 |
| (10% CPD-1) | | | 60 min | 0.36 | 0.68 | 1.38 |

TABLE 7
Effect of Surfactant Type and Concentration

| | Operating Conditions | | | | | | |
|---|---|---|---|---|---|---|---|
| | Operating | Back Pressure | Cycles (c)/Duration | | LD | | |
| Lot No. | Pressure (1000 psig) | (1000 psig) | (min) | | d10 | d50 | d90 |
| TD0680503 | 45 | 3 | 0 | min | 3.16 | 15.92 | 47.07 |
| (0.01% SLS) | | | 1 | cycle | 3.71 | 9.01 | 16.84 |
| | | | 20 | min | 1.79 | 5.97 | 12.8 |
| | | | 40 | min | 0.89 | 3.58 | 10.16 |
| | | | 60 | min | 0.58 | 2.57 | 9.57 |
| TD0690503 | 45 | 3 | 0 | min | 4.15 | 18.26 | 80.39 |
| (0.1% SLS) | | | 1 | cycle | 1.56 | 5.98 | 17.15 |
| | | | 20 | min | 0.65 | 2.16 | 8.02 |
| | | | 40 | min | 0.51 | 1.46 | 5.08 |
| | | | 60 | min | 0.46 | 1.23 | 2.85 |
| TD0700503 | 45 | 3 | 0 | min | 4.02 | 19.29 | 59.6 |
| (1% SLS) | | | 1 | cycle | 1.93 | 6.72 | 17.96 |
| | | | 20 | min | 0.81 | 2.05 | 7.24 |
| | | | 40 | min | 0.43 | 1.12 | 3.31 |
| | | | 60 | min | 0.42 | 0.95 | 2.22 |

TABLE 8
Target Tablet Weight, Hardness, and Disintegration Time

| | | Target Hardness | | | | Disintegration Time | | | | Tablet Weight | | | |
|---|---|---|---|---|---|---|---|---|---|---|---|---|---|
| | | (kp) | | | | (s) | | | | (mg) | | | |
| Active | Lot No. | 5 | 10 | 15 | 20 | 5 | 10 | 15 | 20 | 5 | 10 | 15 | 20 |
| CPD-1 | TD0820603 | 5.4 | 11.0 | 15.3 | 21.9 | 12 | 19 | 138 | 320 | 503 | 506 | 504 | 502 |
| Naproxen | TD0900703 | 4.9 | 9.5 | 14.9 | 19.8 | 6 | 11 | 30 | 125 | 752 | 760 | 751 | 759 |
| (GG01) | | | | | | | | | | | | | |
| Naproxen | TD0980803 | 5 | 10 | 15 | 20 | 10 | 12 | 34 | 45 | 750 | 760 | 759 | 745 |
| (22704HB) | | | | | | | | | | | | | |

TABLE 9
Experimental Conditions for Celecoxib

| | | | | | | | | | | | Operating Conditions^{c} | | |
|---|---|---|---|---|---|---|---|---|---|---|---|---|---|
| | | | | | | | Cell | Seal | Nozzle | | Operating | Back | |
| | | Drug | | | Cell | No. | Diam- | Diam- | Diam- | | Pressure | Pressure | |
| Exp | | Substance | Surfactant | Stabilizer | Set- | of | eter | eter | eter | d50 | (1000 | (1000 | |
| No | Lot No. | (% w/w) | (% w/w) | (% w/w) | Up^{a,b} | Cells | (mm) | (mm) | (mm) | (nm) | psig) | psig) | Cycles(C) |
| 1 | TD2270104 | 20% | 3% HPC-SL | 0.15% SLS | RF | 6 | 0.5 | 2.6 | 0.10 | 218.2 | 45 | 3 | 300 |
| 2 | TD2530104 | 20% | 1% HPC-SL | 0.15% SLS | RF | 6 | 0.5 | 2.6 | 0.13 | 215 | 45 | 3 | 300 |
| 3 | TD2590104 | 20% | 1% HPC-SL | 0.15% SLS | RF | 6 | 0.5 | 2.6 | 0.13 | 206.7 | 45 | 3 | 300 |
| 4 | TD2600104 | 20% | 1% PVP | 0.15% SLS | RF | 6 | 0.5 | 2.6 | 0.13 | 205.2 | 45 | 3 | 90 |
| 5 | 86261 × 52 | 20% | 1% HPC-SL | 0.15% SLS | RF | 11 | 0.5 | 2.6 | 0.25 | 215 | 24 | 0 | 300 |
| 6 | 86261 × 74 | 30% | 1.5% HPC-SL | 0.23% SLS | PF | 11 | 1 | 2.6 | 0.13 | 209 | 45 | 0 | 150 |
| 7 | 86261 × 76 | 30% | 1.5% HPC-SL | 0.23% SLS | PF | 11 | 1 | 2.6 | 0.20 | 195 | 30 | 0 | 120 |
| 8 | 86261 × 77 | 20% | 1.5% | 0.3% DSS | PF | 11 | 1 | 2.6 | 0.13 | 183 | 45 | 0 | 150 |
| | | | copolyvidone | | | | | | | | | | |
| 9 | 86261 × 97 | 20% | 2% PVP | 0.1% SLS | RF | 11 | 0.5 | 2.6 | 0.10 | 215 | 45 | 0 | 90 |
| 10 | 86261 × 99 | 30% | 3% PVP K30 | 0.1% SLS | RF | 6 | 0.5 | 2.6 | 0.10 | 213 | 35 | 0 | 150 |
| 11 | 86261 × 101 | 50% | 5% PVP K30 | 0.27% SLS | PF | 11 | 1 | 2.6 | 0.13 | 224 | 45 | 0 | 150 |

Note
^{a}Turbulent or laminar coupling employed
^{b}RF: Reverse Flow Set-Up; PF: Parallel Flow Set-Up
^{c}Temperature 0-10° C.

What is claimed is:

1. A system for preparing a pharmaceutical granulation, the system comprising:
a high pressure spray homogenizer adapted to receive an active pharmaceutical ingredient and a liquid carrier and to discharge a dispersion, the high pressure spray homogenizer configured to comminute the active pharmaceutical ingredient into solid particles having a median particle size of about 1 μm or less based on volume and to disperse the solid particles in the liquid carrier so as to form the dispersion, wherein the solid particles comprise more than 2% w/w of the dispersion; and
a granulator in fluid communication with the high pressure spray homogenizer and with one or more sources of pharmaceutically acceptable excipients, the granulator configured to receive the dispersion from the high pressure spray homogenizer and to combine the dispersion with the one or more pharmaceutical excipients so as to form the pharmaceutical granulation.

2. The system of claim 1, wherein the high-pressure spray homogenizer is adapted to disperse the solid particles in the liquid carrier so that the solid particles comprise up to about 80% w/w of the dispersion.

3. The system of claim 1, wherein the high-pressure spray homogenizer is adapted to disperse the solid particles in the liquid carrier so that the solid particles comprise 5% w/w or more, 10% w/w or more, 20% w/w or more, 30% w/w or more, 40% w/w or more, 50% w/w or more, 60% w/w or more, or 70% w/w or more of the dispersion.

4. The system of claim 1, wherein the high-pressure spray homogenizer includes a cooling system that permits processing at temperatures below room temperature.

5. The system of claim 1, wherein the high-pressure spray homogenizer includes a cooling system that permits processing at a temperature ranging from about the freezing point of the liquid carrier to about 0° C. or about 10° C.

6. A method of preparing a pharmaceutical granulation, the method comprising:
comminuting an active pharmaceutical ingredient into solid particles in the presence of a liquid carrier so as to form a dispersion, the solid particles having a median particle size of about 1 μm or less based on volume and being substantially insoluble in the liquid carrier at room temperature;
combining the dispersion with one or more pharmaceutically acceptable excipients in a granulator so as to form a pharmaceutical granulation; and
optionally drying the pharmaceutical granulation.

7. A method of preparing a pharmaceutical dispersion, the method comprising comminuting an active pharmaceutical ingredient into particles in the presence of a liquid carrier, the active pharmaceutical ingredient being a solid at room temperature and comprising more than 2% w/w of the pharmaceutical dispersion, and the particles having a median particle size of about 1 μm or less based on volume.

8. The method of claim 6, wherein the particles comprise up to and including about 80% w/w of the dispersion.

9. The method of claim 6, wherein the particles comprise 5% w/w or more, 10% w/w or more, 20% w/w or more, 30% w/w or more, 40% w/w or more, 50% w/w or more, 60% w/w or more, or 70% w/w or more of the dispersion.

10. The method of claim 6, wherein the active pharmaceutical ingredient is comminuted into particles at below room temperature.

11. The method of claim 6, wherein the active pharmaceutical ingredient is comminuted into particles at a temperature ranging from the freezing point of the liquid carrier to a temperature of about 0° C. or 10° C.

12. A pharmaceutical dispersion comprising:
an active pharmaceutical ingredient comprised of particles having a median particle size of about 1 μm or less based on volume;
a liquid carrier; and
an optional surfactant;
wherein the active pharmaceutical ingredient is a solid and is substantially insoluble in the liquid carrier at room temperature and comprises more than 2% w/w of the pharmaceutical dispersion.

13. A method of making a pharmaceutical dosage form, the method comprising:
comminuting an active pharmaceutical ingredient into solid particles in the presence of a liquid carrier so as to form a dispersion, the solid particles having a median particle size of about 1 μm or less based on volume;
combining the dispersion with one or more pharmaceutically acceptable excipients in a granulator so as to form a granulation;
optionally drying the granulation and milling the dried granulation; and
optionally combining the granulation with one or more pharmaceutically acceptable excipients.

14. A method of making a pharmaceutical dosage form, the method comprising:
comminuting an active pharmaceutical ingredient into solid particles in the presence of a liquid carrier so as to form a dispersion, the solid particles having a median particle size of about 1 μm or less based on volume, being substantially insoluble in the liquid carrier at room temperature, and comprising more than 2% w/w of the dispersion; and
combining the dispersion with one or more pharmaceutically acceptable excipients.
